(19) United States
(12) Patent Application Publication
(10) Pub. No.: US 2020/0275852 A1
(43) Pub. Date: Sep. 3, 2020

(54) A SYSTEM, METHOD, COMPUTER PROGRAM AND COMPUTER INTERFACE FOR ANALYSING ELECTROENCEPHALOGRAPHIC INFORMATION

(71) Applicant: KAOSKEY PTY LIMITED, New South Wales (AU)

(72) Inventors: Armen Sargsyan, New South Wales (AU); Dmitriy Melkonian, New South Wales (AU)

(21) Appl. No.: 16/643,751

(22) PCT Filed: Aug. 28, 2018
(86) PCT No.: PCT/AU2018/050918

§ 371 Date: Mar. 2, 2020

(30) Foreign Application Priority Data
Aug. 29, 2017 (AU) 2017903481

(51) Int. Cl. A61B 5/048; A61B 5/00; G16H 50/20; G16H 50/30

(52) U.S. Cl. CPC A61B 5/048; A61B 5/4094; A61B 5/743; G16H 50/30; A61B 5/7257; G16H 50/20; A61B 5/7435

(57) ABSTRACT

A computer enabled method for analysing an electroencephalographic (EEG) waveform to detect the presence of a waveform indicative of an epileptic seizure. The method comprises the steps of, performing a time frequency analysis of the EEG waveform utilising a moving window to perform the analysis on a segment and calculating a power spectrum value of the analysed segment of the waveform to derive an index value. The calculated index value is utilised to determine the presence of a seizure. In a specific embodiment, the invention is directed to the detection of epileptic seizures in rats, mice and other rodents.

### TECHNICAL FIELD OF INVENTION

The present invention relates to a system, method, computer program and interface for analysing electroencephalographic information. In one embodiment, the invention is directed to the use of electroencephalographic information to detect epileptic seizures, spike-and-wave discharges, oscillations (including high-frequency oscillations) and other epileptogenic activity in a mammal, preferably an experimental animal. In a specific embodiment, the invention is directed to the detection of epileptic seizures in rats, mice and other rodents.

### BACKGROUND

Rat models are instrumental in developing theories on the origin of epilepsy, and moreover in the evaluation of new and experimental treatments, as well as in developing new methods for automatic seizure detection and/or prediction. In other words, rat models are commonly used to conduct further research into epilepsy.

However, monitoring for seizures is a laborious and difficult task. Rats and mice are monitored for extended periods that may last several days or even weeks or months, and it is common for researchers to spend several hours reviewing recorded Electroencephalogram (EEG) signals and/or viewing recorded video footage of rats or mice to log or otherwise record the number of seizures experienced by a rat or mouse over the extended period. Such work is laborious. This is particularly the case in situations where several animals are monitored simultaneously which, in the case of prolonged EEG and/or video recordings, is a very time-consuming and tedious process that is prone to error.

Many researchers continue to harbour a preference to identify seizures by reviewing the video footage and visually inspecting the recorded EEG signal, as “automated” detection methodologies available to date have been prone to unacceptable levels of error.

It is with these problems in mind that the present invention has been developed.

### SUMMARY OF THE INVENTION

In a first aspect, there is provided a computer enabled method for analysing an electroencephalographic (EEG) waveform to detect the presence of a waveform indicative of an epileptic seizure, comprising the steps of, performing a time frequency analysis of the EEG waveform, utilising a moving window to perform the analysis on a segment of the waveform, calculating a power spectrum value of the analysed segment of the waveform to derive an index value, wherein the index value is utilised to determine the presence of a seizure.

One embodiment includes the further step of iterating the process steps of the first aspect of the invention to provide a plurality of index values for different time windows, wherein each of the plurality of index values is utilised to detect a seizure.

One embodiment includes the step of sorting the plurality of index values into a histogram, wherein the resultant histogram is analysed to define a background level of activity, wherein the background level of activity defines a threshold value utilised to remove background activity.

In one embodiment, the method comprises the step of providing an interface to allow a user to review the histogram and selectively reset the threshold value.

The method may comprise the step of autonomously reviewing the selective resets of the threshold value by a user, and varying the predetermined threshold value on the basis of the selective resets.

The method may comprise the further step of analysing the EEG waveform only within a defined frequency band.

The defined frequency band for rats preferably extends from 17 to 25 Hz. The defined frequency band for spike-and-wave detection in mutant mouse models is slightly wider, preferably extending from 14-27 Hz.

The method may be optimised for the detection of epileptic seizures in rats, mice and other rodents.

In a second aspect, there is provided a system for analysing an electroencephalographic (EEG) waveform to detect the presence of an activity pattern indicative of an epileptic seizure, comprising, a module arranged to utilise a processor to perform a time frequency analysis of the EEG waveform, utilising a moving window to perform the analysis on a segment, the module calculating a power spectrum value of the analysed segment of the waveform to derive an index value, and utilising the index value to determine the presence of a seizure pattern in the waveform, wherein the presence of a seizure pattern is communicated to a user via an interface.

In a third aspect, there is provided a computer program incorporating at least one instruction and arranged to, when executed on a computing system, perform the method steps of the first aspect of the invention.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is an example computing system on which a method and/or a computer program may be operated, in accordance with an embodiment of the invention;

FIG. 2 is a flowchart illustrating a computer enabled method in accordance with an embodiment of the invention;

FIG. 3 is a graph illustrating an EEG signal and a window power spectrum calculated in accordance with an embodiment of the invention;

FIG. 4 is a graph illustrating a curve of a Spectral Band Index (SBI) in accordance with an embodiment of the invention;

FIG. 5a is a SBI curve with an automatically calculated threshold as displayed on an interface in accordance with an embodiment of the invention;

FIG. 5b is a SBI histogram with the same threshold, as displayed on an interface in accordance with an embodiment of the invention; and

FIGS. 6a and 6b are screen shots illustrating input signal and results windows, respectively, of an interface of a computer program in accordance with an embodiment of the invention.

### DETAILED DESCRIPTION OF PREFERRED EMBODIMENTS

The present invention relates generally to a system, method, computer program and interface for analysing electroencephalographic information. In one embodiment, the invention is directed to the use of electroencephalographic information to detect epileptic seizures in a mammal. In one embodiment which is described in more detail herein below, the invention is directed to the detection of epileptic seizures in rats, mice and other rodents.

In more detail, one aspect of the embodiments described herein provides a method for analysing electroencephalographic information in either an on-line (i.e. analysis performed in real time, as data is being collected) or off-line (i.e. analysis performed based on pre-recorded/saved data) manner. In the case of on-line analysis, the method comprises the steps of performing a time frequency analysis of an ongoing EEG signal, calculating a power spectrum value of the analysed segment of the signal to derive an index value, wherein the index value is utilised to determine the presence of a seizure. In the case of off-line analysis, apart from the one difference, namely that EEG signal data is read from a file rather than being received as an ongoing signal, the same method steps are performed.

Such a system is particularly useful for situations where the amount of data that needs to be processed is voluminous, and there is a need to process large amounts of data in an autonomous manner, while minimising “false positives”. That is, events that are not true events, but events that are artefacts of the signal collection process, such as electrical interference.

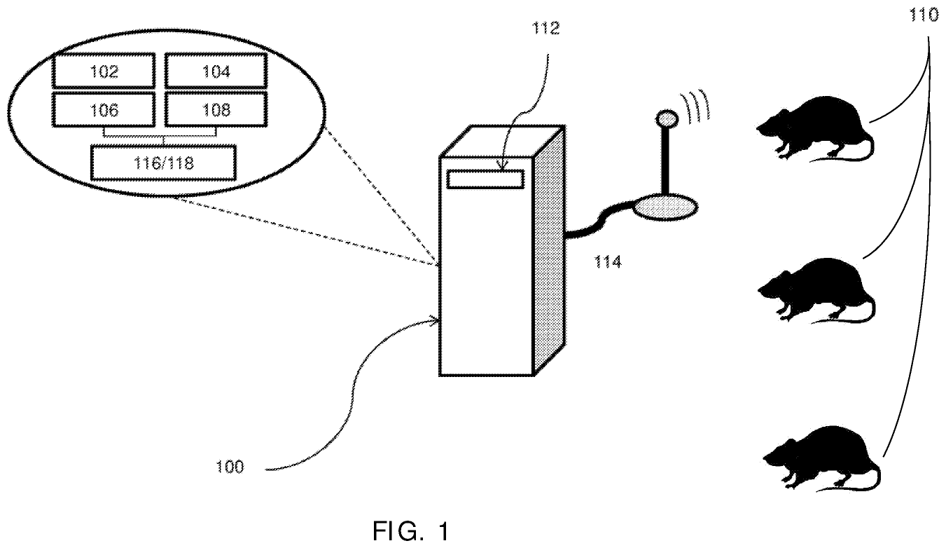

One embodiment of the method is codified in a computing system, such as the computing system shown at FIG. 1.

In FIG. 1 there is shown a schematic diagram of a computing system, which in this embodiment is a computing system 100 suitable for use with an embodiment of the present invention. The computing system 100 may be used to execute application and/or system services such as a computer program and an interface in accordance with an embodiment of the present invention.

With reference to FIG. 1, the computing system 100 may comprise suitable components necessary to receive, store and execute appropriate computer instructions. The components may include a processor 102, read only memory (ROM) 104, random access memory (RAM) 106, an input/output device such as a disc drive 108, a remote or connected input device 110 (such as an EEG signal monitor), and one or more communications link(s) 114.

The computing system 100 includes instructions that may be installed in ROM 104, RAM 106 or disc drives 108 and may be executed by the processor 102. There may be provided a plurality of communication links 114 which may variously connect to one or more input devices 110, such as EEG signal monitors. At least one of a plurality of communications link 114 may be connected to an external computing network through a telecommunications network.

In one particular embodiment the device may include a database 116 which may reside on the storage device 112. It will be understood that the database may reside on any suitable storage device, which may encompass solid state drives, hard disc drives, optical drives or magnetic tape drives. The database 116 may reside on a single physical storage device or may be spread across multiple storage devices, either locally or remotely.

The computing system 100 includes a suitable operating system 118 which may also reside on a storage device or in the ROM of the server 100. The operating system is arranged to interact with the database and with one or more computer programs to cause the server to carry out the steps, functions and/or procedures in accordance with the embodiments of the invention described herein.

Broadly, the invention relates to a computing method and system arranged to receive data from one or more remote devices via a communications network. The remote devices may take the form of data stored on a computing device, but may also take the form of signal data received directly from a recorder. In the instance of signal data, the data takes the form of an analogue wave, namely a “raw” Electroencephalogram (EEG) signal, as will be described in more detail later. The remote device may also take the form of a camera, to capture image or video data, as will also be described in more detail later.

In the ensuing description, the broader invention will be described with reference to a particular application, namely the detection of epileptic seizures in experimental animals, and rodents in particular. All data described herein relates to experiments carried out with rats and mice.

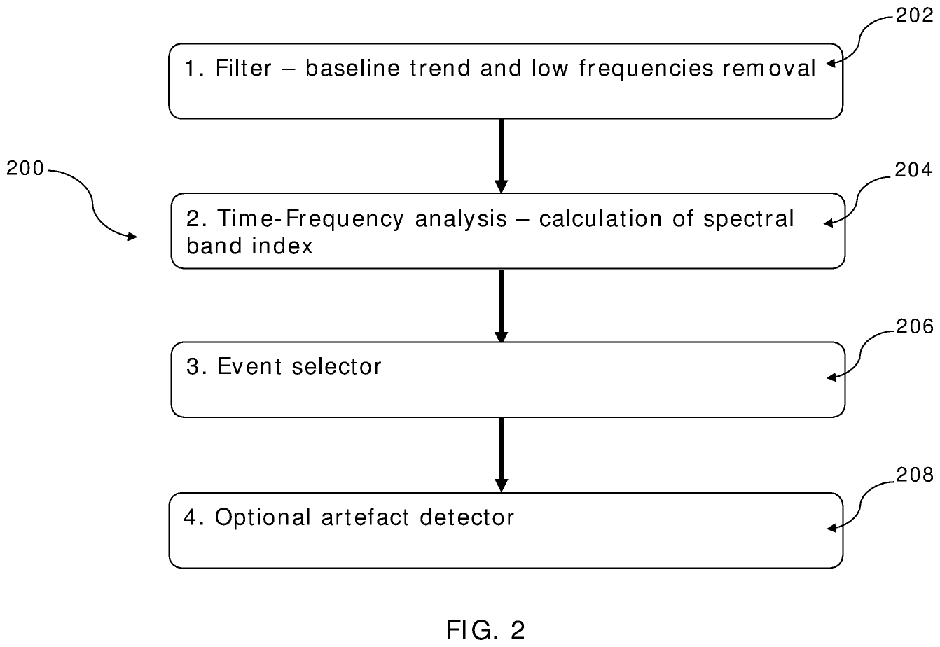

Turning to FIG. 2, there is described an automated seizure detection algorithm 200 in accordance with an embodiment of the present invention.

It has been found that an epileptic seizure produces a regular and distinguishable pattern in the EEG signals (which is representative of electrical activity) produced by the brain of a mammal. For example, in the article by Colin D. Binnie and Hermann Stefan, entitled “Modern electroencephalography: its role in epilepsy management”, published in Clinical Neurophysiology 110 (1999) at pages 1671-1697, there is provided an overview of EEG signals, measurement techniques and methodologies, and findings regarding the significance or otherwise of patterns in EEG signals, as they pertain to candidates who have epilepsy. The entire contents of the abovementioned paper is incorporated herein by reference. The embodiment described herein utilises a signal processing algorithm to detect the presence of such signals in collected EEG data.

At step 202, an incoming signal is analysed, and a filter is applied to remove any low frequency baseline trend signals, which, for the intended purpose of the embodiment described herein (namely the detection of epileptic seizures), do not contain immediately relevant information regarding epileptic seizures. As an example, in the embodiment described herein, where the application is directed to detecting seizures in rats and mice, low frequency components which are of no interest clinically for detecting seizures are automatically removed, and components in the frequency range of interest are made more recognizable.

At step 204, a time frequency analysis is performed utilising a Similar Basis Function (SBF) algorithm. For each window, the power spectrum of the window signal is calculated, using the SBF algorithm. A more detailed explanation of the SBF algorithm is provided in the paper by Dmitriy Melkonian, entitled “Similar basis function algorithm for numerical estimation of Fourier integrals”, published in Numerical Algorithms (2010) 54 at pages 73-100. The entire contents of the abovementioned paper is incorporated herein by reference. The power spectrum is calculated only within the frequency band of interest (in the embodiment, 17-25 Hz) thereby significantly reducing the number of calculations. The frequency resolution utilised is arbitrary, being either pre-set, or defined by the user. The application of the SBF algorithm and the resultant output data produces a “Spectral Band Index curve” (SBI curve) with specific peaks.

In more detail, the algorithm applies a time window of fixed (user-defined) size along the EEG signal, using a user-defined step size. The step size used in the examples provided herein is equal to the half of the window size.

Once the power spectrum is calculated, a measure (in the form of a single real number) is estimated, the measure being indicative of the spectral intensity within the frequency band. This measure is termed the “Spectral Band Index” value (SBI value).

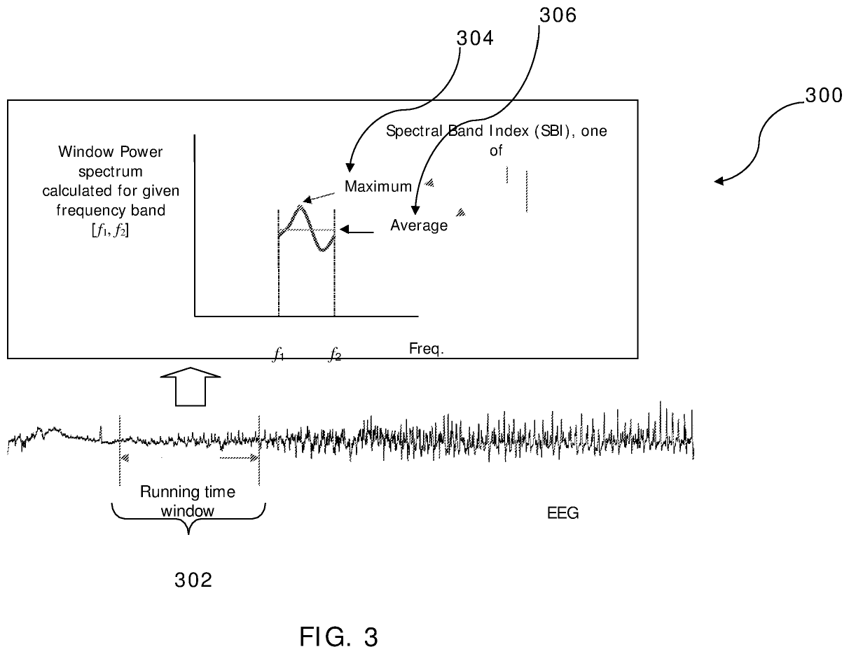

The manner in which the SBI value is calculated may vary, as the SBI value is a relative measure not an absolute. For example, the maximum value within the frequency band may be utilised, or the calculated area under the power spectrum within the frequency band may also be utilised. In testing by the inventors, both measures (the maximum and the area) can be utilised. However, it has been determined that use of the maximum value is preferable in the case where there is a narrow peak of the power spectrum within the frequency band of interest, as shown in FIG. 3, wherein the SBI value data shown generally at 300, which is determined by either the maximum measure 304 or area measure 306, utilising the window 302.

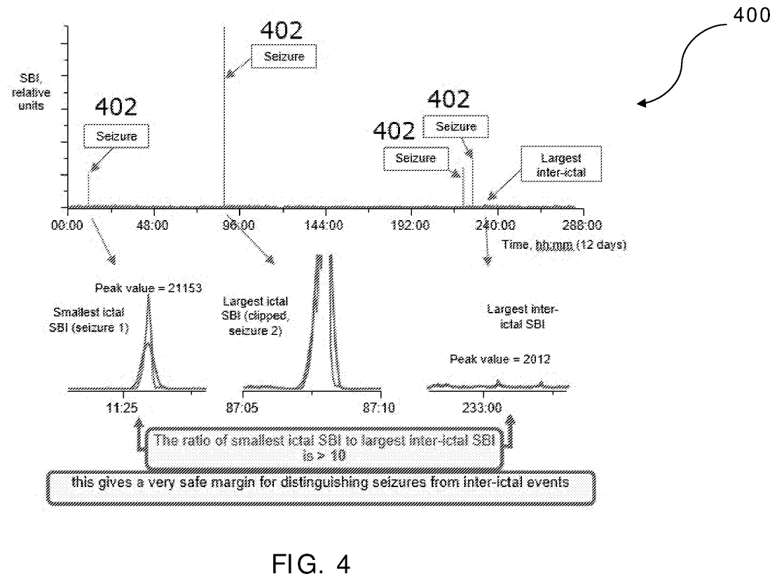

The value of the measure is plotted against the centre of the window's time value to form the SBI curve (dependence of the measure on time) as shown in FIG. 4. Optionally, a smoothing algorithm may be applied to the SBI curve, to unite nearby sharp peaks above the threshold (which are caused by the same event) into one value and thereby reduce the number of selected events. The SBI value provides a very safe margin for distinguishing seizures from other events, such as those identified at 402.

Referring again to FIG. 2 at step 206, a threshold value is automatically applied to the SBI curve, to discount the subthreshold SBI values that correspond to interictal (or background, or normal) EEG activity, wherein “ictal events” are seizure events and “interictal” is a term that refers to the time between seizure events. The remaining suprathreshold peaks in the SBI curve are identified as seizure events.

In more detail, a threshold value of the SBI curve is defined, such that background or other non-seizure related activity lies below the threshold. To define this threshold, a distribution histogram of the SBI values is calculated. Assuming that the total duration of ictal events is only a small fraction of overall duration of the record, the region of the histogram with highest density of distribution will indicate the interictal SBI value range. By setting the threshold somewhere above this range (at the right edge or higher) the “ordinary” (i.e. non relevant) values of the SBI are removed and only the outstanding values are left in the histogram.

In more detail (and referring to the computer program when it is operating in off-line mode), the distribution histogram of SBI values is firstly built over the entire recording time.

If the number of bins of the histogram is N and hᵢ is the value of the i-th bin, then the process begins by finding the maximum of the histogram and corresponding bin iₘₐₓ.

Starting from the maximal bin the algorithm scans subsequent bins in the histogram to the right of the maximal bin to find a bin where the value of the bin is almost zero. For this, starting from iₘₐₓ the sum of K consecutive bins is calculated:

$${S}_{j} = {{\sum }_{k = {i}_{max} + j}}^{{i}_{max} + j + K} {h}_{k}$$

Where j=0, 1, 2, . . . and for each j the condition Sⱼ<A is determined, where A is a parameter described in more detail below. As soon as this condition is satisfied, the corresponding bin's x-value is taken as the threshold (the x-values of the histogram are the SBI amplitudes).

The values for parameters used in the validation study were K=N/20 and A=K/2, where K is the number of consecutive bins.

In other words, the sum of bins must be not greater than half of the total number of bins. Put another way, at least half of the bins must be empty. In practice, the inventors have found that this requirement is stringent, and is only satisfied for long, artefact-free recordings with few seizures.

Therefore, the parameters K and A can be modified during learning or adaptation of the algorithm. In this regard, the software has a “learning” capability, wherein user adjustment is saved and an algorithm is applied to vary the parameters K and A based on user input.

In an online mode the threshold is calculated with the same underlying methodology, the only difference being that the histogram is recalculated in real-time in each instance where a new data window is acquired and a corresponding SBI value is calculated. Therefore, it follows that the threshold is redefined dynamically with each new SBI value.

At step 208, a user may optionally review the raw EEG signal and/or video footage, to confirm that the peaks are in fact true events and not artefacts, and may adjust the threshold and/or discount events.

In more detail, with regard to the embodiment described herein, the inventors have found that the seizures in at least in four (4) different rat models of epilepsy have specific frequency component that do not appear (or is very weak compared to seizures) in the interictal EEG in artefact free recordings. This component has peak frequency in the range of 17-25 Hz.

Using the specificity provided by these four models, the embodiment described herein (and the resultant data shown herein) utilises an algorithm that is “fine-tuned” to process the Spectral Band Index (SBI) for the narrow band of frequencies for the four rat models (i.e. 17-25 Hz) and selects the fragments of the EEG where the SBI value is high (above the threshold, which is first determined automatically, then may be adjusted by the user).

The program can operate in a fully autonomous mode, and selects a threshold value in the manner described above. However, as there are often situations where unusual artefacts occur (which may not be caught by the autonomous setting of the threshold value), the embodiment described herein provides an interface which allows for user intervention.

An example of an artefact is a signal that has a strong component in both the desired frequency band and also in all other bands, meaning that it is not a signal indicative of an epileptic seizure, but rather a “noise” signal, such as electrical interference.

It will be understood that the example above has been described with reference to the computer program processing an existing data file (or files) of EEG data. However, the computer program is capable of calculating and populating a histogram in real time, as data is received.

In the case where data is being received in real time, the methodology is identical to the methodology described above with regard to FIG. 2 and steps 202 through to 208, the only difference being that step 204 is iterated as each new discrete data set is received by the program. It will be understood that such variations are within the purview of a person skilled in the art.

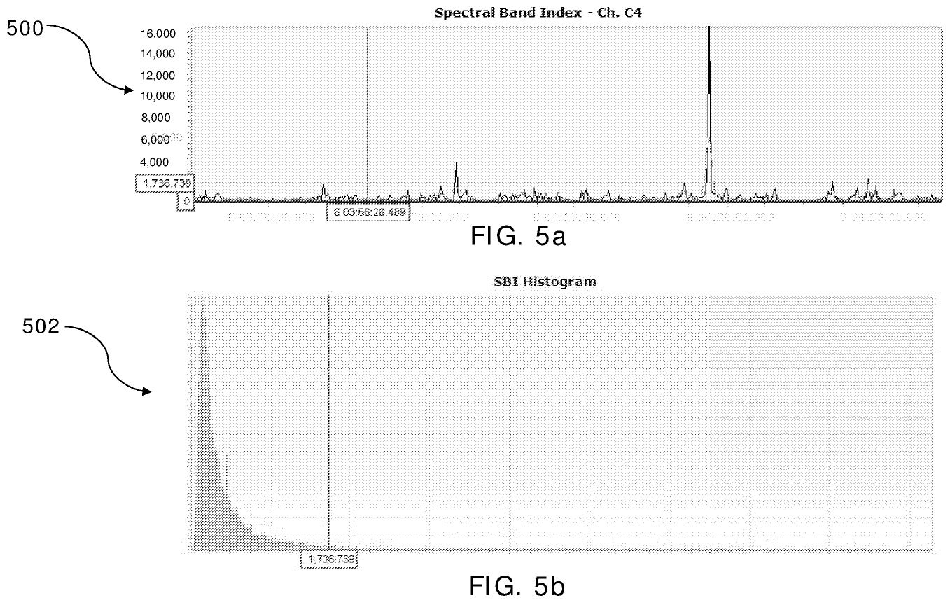

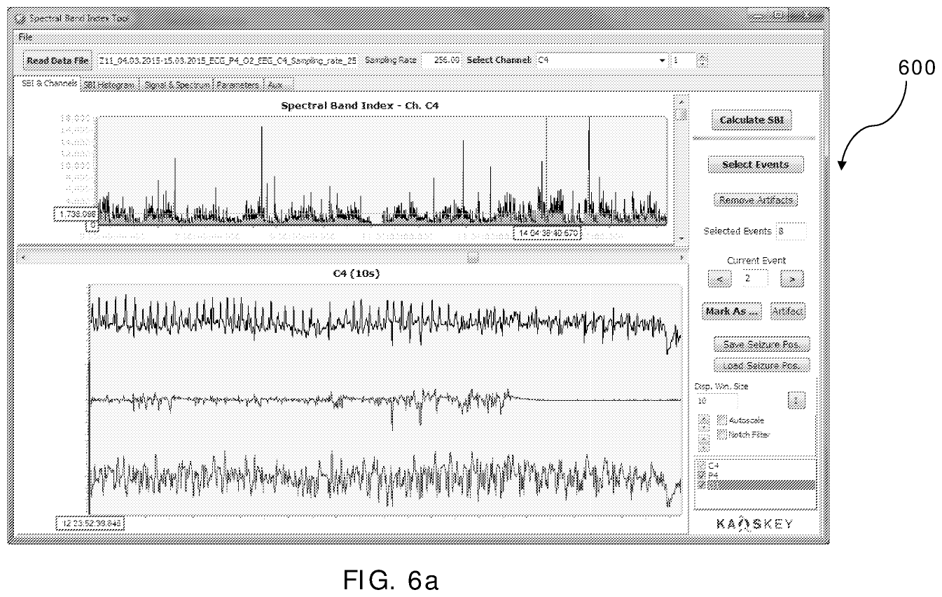

Referring to FIGS. 5a and 5b, there are shown corresponding windows in a user interface, which provide a visual display of the SBI curve and SBI histogram. A user may inspect the data and use a mouse or other pointing device to change the threshold value.

The automatically determined threshold may then be re-adjusted by user, according to some knowledge and experience (e.g., the threshold may be lowered to capture weaker or shorter events). This readjustment is very simple—the user utilises a mouse or other pointing device (e.g. their finger in the case of a touchscreen) to “drag” the threshold line towards the left or the right on the histogram plot, or up or down on the SBI plot.

After the threshold is defined, all episodes of the EEG for which the SBI is above the threshold, are selected in a list of events, with their start and end times. The start and end times are roughly determined as the points where the SBI curve crosses the threshold. The precision of defining the starts and ends in this way is equal to the size of time window. These values may then be automatically redefined with more precision, by using a smaller time window.

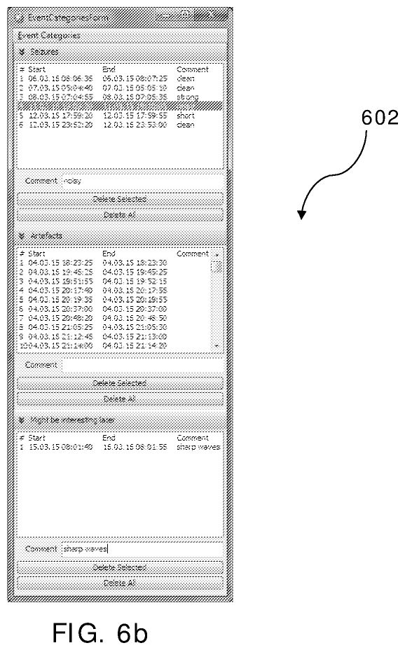

The user then may “leaf” through the selected events (viewing the corresponding fragment of the EEG) and confirm or reject the event as a seizure. The confirmed events form a new list of events. After finishing the inspection, user may save the final list of events. Such changes can be effected through interfaces 600 and 602 in FIGS. 6a and 6b, respectively.

Moreover, while the preceding description has described the computer program in accordance with the embodiment providing a means for dividing signals into “events” and “non-events”, the program also has the functionality to allow the user to add an arbitrary number of event types (or categories) to create a more detailed classification of the events.

That is, the computer program provides two default event categories (in other words, two lists, namely a first list entitled “Seizures”, and a second list entitled “Artefacts”), but the user may add additional categories.

The user cannot delete the two lists, but may rename the lists and provide additional description. Moreover, the user may add any number of additional lists, or event categories. The added categories may be deleted. When inspecting the automatically selected events, the user can add an event into any one of these event categories (lists). The example shown in FIG. 6b shows three (3) categories: two (2) default (“Seizures” and “Artefacts”) and one added (entitled “May be interesting later”).

This feature is useful where a user identifies an event that they cannot classify as either a seizure or as an artefact, but that they do not want to discard—the user can create an additional “unknown” category to categorise such events. Or, when the user wants to differentiate the seizures according to some criteria (e.g. duration or clinical/subclinical), in which case they can create corresponding categories and place events into the categories as they require.

### Advantages

The embodiment and broader invention described herein provides a number of advantages.

Firstly, using an optimized algorithm allows for more precise frequency characteristics (no spectral leakage) and for arbitrary frequency range and resolution. For example, no 50 Hz or 60 Hz power noise filtering is required when the range of interest does not include 50 or 60 Hz.

Secondly, only characteristics in the frequency range of interest are calculated, reducing the number of calculations required, and thereby increasing the speed of calculation in a manner that is comparable in speed with a Fast Fourier Transform.

In addition, the algorithm is optimized for window-wise calculation (using a running window to calculate the Spectral Band Index), so that the heavy processing required to compute sine and cosine coefficients is performed only once and the coefficients are re-used for each subsequent window, which also significantly increases the calculation speed.

The system, method and computer program are arranged to process results in a more efficient and accurate manner. In order to demonstrate the efficiency and accuracy of the computer program, the applicant conducted a short comparison study of a traditional, prior art method of identifying seizures, and the computer program of the embodiment.

EEG signals from one hundred and seventy nine (179) rats and twenty six (26) mice were processed utilising both the embodiment of the invention, and the prior art method of manually reviewing EEG signals and video recordings. As shown in Table 1 (below), the rat records contained 10,600 seizures in total, and the mice records contained 8,566 seizures in total.

The computer program detected 100% of all seizures, in all records, and in all models. In several cases the program found seizures that were not identified at first pass by an expert. These events were verified afterwards by the expert and were confirmed to be seizures. As such, the algorithm utilised by the computer program is more accurate in detecting seizures than a human manually viewing footage or reviewing EEG signals.

TABLE 1
Processing results

| | No. of | Number of Seizures | | |
|---|---|---|---|---|
| Model | Animals | Annotated | Detected | Verified |
| Rats | | | | |
| Post-SE | 119 | 989 | 993 (+4) | 993 |
| Post-status epilepticus model | | | | |
| of temporal lobe epilepsy | | | | |
| PTE | 5 | 43 | 49 (+6) | 49 |
| Fluid percussion injury model | | | | |
| of post-traumatic epilepsy | | | | |
| GAERS | 41 | 8733 | 8733 | |
| Genetic Absence Epilepsy Rat | | | | |
| from Strasbourg | | | | |
| WAG/Rij | 14 | 825 | 825 | |
| Wistar Albino Glaxo/Rijswijk | | | | |
| model of absence epilepsy | | | | |
| Total for rats | 179 | 10590 | 10600 | |
| Mice | | | | |
| Mutant mouse model on a | 4 | 55 | 55 | |
| variant of strain backgrounds | | | | |
| in the gene Gabrg2 | | | | |
| Mutant mouse model on a | 8 | 601 | 601 | |
| variant of strain backgrounds | | | | |
| in the gene Gria4 | | | | |
| Mutant mouse model on a | 4 | 453 | 453 | |
| variant of strain backgrounds | | | | |
| in the gene Scn8a | | | | |
| Mutant mouse model on a | 2 | 7367 | 7367 | |
| variant of strain backgrounds | | | | |
| in the gene Gnb1 | | | | |
| Kcnt1 gene knockin mouse | 8 | 90 | 90 | |
| model | | | | |
| Total for mice | 26 | 8566 | 8566 | |
| Total (rats and mice) | 205 | 19156 | 19166 | |

Moreover, substantive time efficiencies are achieved by utilising the computer program of the embodiment to review EEG signal data. Referring to Table 2 (below), the longest amount of time spent by a user, when utilising the computer program to process one day's worth of data for one (1) Post-SE or PTE animal was approximately five (5) minutes, whereas the shortest time spent by a user, when utilising the computer program to process data for one (1) animal was approximately six (6) seconds. Correspondingly, including the amount of time needed by the user to interact with the interface of the computer program to review the identified events (where there was user input required to adjust or vary the threshold and/or remove artefacts), the average amount of time spent by user, utilising the computer program to process one (1) day's worth of data for one (1) animal was approximately one (1) minute.

An experienced researcher, it was found, when not using the computer program in accordance with the embodiment of the invention, spends approximately one (1) hour scrolling through one (1) day's worth of EEG data for one (1) animal to identify all seizures experienced by the animal.

Thus, using the computer program in accordance with an embodiment of the invention, researchers reduce, on average, the time required for analysis by a factor of sixty (60), thereby saving around 98% of time.

TABLE 2
Processing time for Post-SE and PTE rats per method

| | | |
|---|---|---|
| Max processing time per 1 day record | 5 | minutes |
| Min processing time per 1 day record | 0.1 | minutes |
| Average processing time per 1 day record | 1 | minute |
| Approximate time spent by expert | 60 | minutes |
| for visual examination of 1 day record | | |

| | |
|---|---|
| Average reduction of processing time | 60 times - saving |
| | 98.3% of time |

### Disclaimers

Throughout this specification, unless the context requires otherwise, the word “comprise” or variations such as “comprises” or “comprising”, will be understood to imply the inclusion of a stated feature or group of features but not the explicit exclusion of any other feature or group of features.

Those skilled in the art will appreciate that the embodiments described herein are susceptible to obvious variations and modifications other than those specifically described and it is intended that the broadest claims cover all such variations and modifications. Those skilled in the art will also understand that the inventive concept that underpins the broadest claims may include any number of the steps, features, and concepts referred to or indicated in the specification, either individually or collectively, and any and all combinations of any two or more of the steps or features may constitute an invention.

Where definitions for selected terms used herein are found within the detailed description of the invention, it is intended that such definitions apply to the claimed invention. However, if not explicitly defined, all scientific and technical terms used herein have the same meaning as commonly understood to one of ordinary skill in the art to which the invention belongs.

Although not required, the embodiments described with reference to the method, computer program, computer interface and aspects of the system can be implemented via an application programming interface (API), an application development kit (ADK) or as a series of program libraries, for use by a developer, for the creation of software applications which are to be used on any one or more computing platforms or devices, such as a terminal or personal computer operating system or a portable computing device, a smartphone or a tablet computing system operating system, or within a larger server structure, such as a ‘data farm’ or within a larger computing transaction processing system.

Generally, as program modules include routines, programs, objects, components and data files that perform or assist in the performance of particular functions, it will be understood that the functionality of the method, computer program and computer interface defined herein may be distributed across a number of routines, programs, objects or components to achieve the same functionality as the embodiment and the broader invention claimed herein. Such variations and modifications are contemplated by the inventor and are within the purview of those skilled in the art.

It will also be appreciated that where methods and systems of the present invention and/or embodiments are implemented by computing systems or partly implemented by computing systems then any appropriate computing system architecture may be utilised without departing from the inventive concept. This includes standalone computers, networked computers and dedicated computing devices that do not utilise software as it is colloquially understood (such as field-programmable gate arrays).

Where the terms “computer”, “computing system” and “computing device” are used in the specification, these terms are intended to cover any appropriate arrangement of computer hardware for implementing the inventive concept and/or embodiments described herein.

Where the terms “software application”, “application”, “computer program” and “program” are used in the specification when referring to an embodiment of the invention, these terms are intended to cover any appropriate software which is capable of performing the functions and/or achieving the outcomes as broadly described herein.

Where reference is made to communication standards, methods and/or systems, it will be understood that the devices, computing systems, servers, etc., that constitute the embodiments and/or invention or interact with the embodiments and/or invention may transmit and receive data via any suitable hardware mechanism and software protocol, including wired and wireless communications protocols, such as but not limited to second, third and fourth generation (2G, 3G and 4G) telecommunications protocols (in accordance with the International Mobile Telecommunications-2000 (IMT-2000) specification), Wi-Fi (in accordance with the IEEE 802.11 standards), Bluetooth (in accordance with the IEEE 802.15.1 standard and/or standards set by the Bluetooth Special Interest Group), or any other radio frequency, optical, acoustic, magnetic, or any other form or method of communication that may become available from time to time.

1. A computer enabled method for analysing an electroencephalographic (EEG) waveform to detect the presence of a waveform indicative of an epileptic seizure, comprising the steps of, performing a time frequency analysis of the EEG waveform utilising a moving window to perform the analysis on a segment, calculating a power spectrum value of the analysed segment of the waveform to derive an index value, wherein the index value is utilised to determine the presence of a seizure.

2. A method in accordance with claim 1, further comprising the step of iterating the process steps of claim 1 to provide a plurality of index values for different time windows, wherein each of the plurality of index values is utilised to detect a seizure.

3. A method in accordance with claim 2, comprising the step of sorting the plurality of index values into a histogram, wherein the resultant histogram is analysed to define a background level of activity, wherein the background level of activity defines a threshold value utilised to remove background activity.

4. A method in accordance with claim 3, further comprising the step of providing an interface to allow a user to review the histogram and selectively reset the threshold value.

5. A method in accordance with claim 4, further comprising the step of autonomously reviewing the selective resets of the threshold value by a user, and varying the predetermined threshold value on the basis of the selective resets.

6. A method in accordance with claim 1, further comprising the step of analysing the EEG waveform only within a defined frequency band.

7. A method in accordance with claim 6, wherein the defined frequency band for rats extends from 17 to 25 Hz, and the defined frequency band for mice extends from 14 to 27 Hz.

8. A method in accordance with claim 1, wherein the method may be optimised for the detection of epileptic seizures in rats and mice.

9. A system for analysing an electroencephalographic (EEG) waveform to detect the presence of an activity pattern indicative of an epileptic seizure, comprising, a module arranged to utilise a processor to perform a time frequency analysis of the EEG waveform utilising a moving window to perform the analysis on a segment, the module calculating a power spectrum value of the analysed segment of the waveform to derive an index value, and utilising the index value to determine the presence of a seizure pattern in the waveform, wherein the presence of a seizure pattern is communicated to a user via an interface.

10. A computer program incorporating at least one instruction and arranged to, when executed on a computing system, perform the method steps of claim 1.
